(19) United States
(12) Patent Application Publication
(10) Pub. No.: US 2024/0091185 A1
(43) Pub. Date: Mar. 21, 2024

(54) COMPOSITION FOR IMPROVING COGNITIVE FUNCTION, AGENT FOR IMPROVING COGNITIVE FUNCTION, AND FOOD FOR IMPROVING COGNITIVE FUNCTION

(71) Applicant: AJINOMOTO CO., INC., Tokyo (JP)

(72) Inventors: Hidehiro NAKAMURA, Kawasaki-shi (JP); Noriko KAWASAKI, Kawasaki-shi (JP); Yasuko KAWAMATA, Kawasaki-shi (JP); Akihiko KITAMURA, Kawasaki-shi (JP); Ryosei SAKAI, Kawasaki-shi (JP); Fumika TAKEUCHI, Kawasaki-shi (JP)

(73) Assignee: AJINOMOTO CO., INC., Tokyo (JP)

(21) Appl. No.: 18/514,387

(22) Filed: Nov. 20, 2023

Related U.S. Application Data
(63) Continuation of application No. PCT/JP2022/020975, filed on May 20, 2022.

(30) Foreign Application Priority Data
May 21, 2021 (JP) 2021-086454

(51) Int. Cl. A61K 31/198; A61K 31/685; A61P 25/28

(52) U.S. Cl. CPC A61K 31/198; A61K 31/685; A61P 25/28

(57) ABSTRACT

Compositions, containing glycine and serine and having a content ratio of glycine to serine (glycine/serine) of not less than 0.6 in weight ratio are effective for improving cognitive function, and have an effective preventive or improving effect on a decline in cognitive function and cognitive dysfunction, are highly safe, and enable continuous ingestion or administration.

### CROSS REFERENCES TO RELATED APPLICATIONS

This application is a continuation of International Patent Application No. PCT/JP2022/020975, filed on May 20, 2022, and claims priority to Japanese Patent Application No. 2021-086454, filed on May 21, 2021, both of which are incorporated herein by reference in their entireties.

### BACKGROUND OF THE INVENTION

### Field of the Invention

The present invention relates to compositions for improving cognitive function, agents for improving cognitive function, and foods for improving cognitive function.

### Discussion of the Background

With the recent rapid increase in the elderly population, the number of patients with dementia increased rapidly, and it has been estimated that the prevalence of dementia among elderly people aged 65 and over is 16.7% in 2020.

According to the Ministry of Health, Labour and Welfare of Japan, dementia is defined to mean “a state in which various mental functions that once developed normally after the birth have chronically declined and disappeared to the extent that daily life and social life cannot be managed”.

Dementia includes dementia caused by various diseases such as Alzheimer-type dementia, frontotemporal dementia (Pick disease etc.), Lewy body dementia, and cerebrovascular dementia. While aging is the greatest risk factor, its cause is often unclear.

However, in any type of dementia, cognitive dysfunction such as memory disorder, disorientation and the like are observed as core symptoms, and peripheral symptoms such as behavior abnormality, mental symptoms and the like are also commonly observed, and progression of the above-mentioned symptoms has serious effects such as increased burden of nursing care and the like for not only patients but also their families.

Currently, acetylcholinesterase inhibitors such as donepezil hydrochloride, and NMDA (N-methyl-D-aspartate) receptor antagonists such as memantine have been approved as therapeutic drugs for dementia. However, these therapeutic drugs for dementia are basically for Alzheimer-type dementia, are symptomatic treatment drugs, and can merely suppress progression of the symptoms somewhat.

Regarding the prevention of dementia, the importance of exercise and meal in preventing and improving a decline in cognitive function is known.

Regarding exercise, it has been found that an increase in cerebral blood flow due to exercise can improve physical activity and prevent Alzheimer's disease.

Regarding meals, antioxidants such as vitamin C, vitamin E, and β-carotene, ω-3 long-chain unsaturated fatty acid, amino acid, and the like are attracting attention.

Particularly, since amino acid is a nutrient with extensive food experience and abundant in various foods, many attempts have been made to utilize amino acid to prevent and improve a decline in cognitive function. For example, use of L-lysine for the improvement of cognitive function of healthy subjects (see WO 2010/068173, which is incorporated herein by reference in its entirety), a composition for preventing or treating brain function disorders containing at least one kind of L-serine, glycine, and fatty acid compounds thereof (see U.S. Pat. No. 6,310,097B1, which is incorporated herein by reference in its entirety), a composition containing phosphatidyl serine and/or lysophosphatidyl serine, a phospholipid other than those mentioned above, and a serine source such as L-serine, 0-phospho-L-serine, or the like (see JP-A-2007-504197, which is incorporated herein by reference in its entirety) are disclosed.

However, middle-aged and elderly people who are at risk of decline in cognitive function and cognitive dysfunction include many of those who are limited in exercise performance due to disease and the like, and those having difficulty in continuing exercise due to a decline in physical function.

In addition, some of the above-mentioned ingredients such as amino acid and the like that are ingested in meals do not have sufficient preventive or improving effects against a decline in cognitive function and cognitive dysfunction, and many require future verification of the effectiveness. For example, U.S. Pat. No. 6,310,097B1, which is incorporated herein by reference in its entirety, shows that serine and glycine enhance survival activity of hippocampal neurons, glycine suppresses neuronal cell death, and serine enhances the proliferation-promoting effects of tumor necrosis factor-α (TNF-α) and brain-derived neurotrophic factor (BDNF) on Purkinje cells. However, the level of preventing or improving effects achieved by serine and glycine on cognitive function decline or cognitive dysfunction has not been shown. In addition, JP-A-2007-504197, which is incorporated herein by reference in its entirety, does not show specific study results regarding the improving and reinforcing action of the composition described in JP-A-2007-504197, which is incorporated herein by reference in its entirety, on memory functions.

Therefore, a component having an effective preventive or improving effect on the decline in cognitive function and cognitive dysfunction, which is highly safe and can be ingested continuously, has been desired.

### SUMMARY OF THE INVENTION

Accordingly, it is an object of the present invention to provide a composition for improving cognitive function, which has an effective preventive or improving effect on a decline in cognitive function and cognitive dysfunction, is highly safe, and enables continuous ingestion or administration.

It is another object of the present invention to provide novel methods for improving cognitive function.

These and other objects, which will become apparent during the following detailed description, have been achieved by the present inventors' discovery that a composition containing glycine and serine, and having a content ratio of glycine to serine (glycine/serine) of not less than 0.6 in weight ratio is superior in the effects of improving a decline in learning and/or memory functions and learning and/or memory disorders. They have conducted further studies and completed the present invention.

That is, the present invention relates to the following.
(1) A composition for improving cognitive function, comprising glycine and serine and having a content ratio of glycine to serine (glycine/serine) of not less than 0.6 in weight ratio.
(2) A composition for improving cognitive function, comprising glycine and serine and having a content ratio of glycine to serine (glycine/serine) of not less than 2 in weight ratio.
(3) The composition of (1), further comprising one or more kinds selected from the group consisting of phospholipid, glutamic acid, cysteine, and cystine.
(4) The composition of (2), further comprising one or more kinds selected from the group consisting of phospholipid, glutamic acid, cysteine, and cystine.
(5) The composition of any of (1) to (4), for improving a decline in learning and/or memory functions or learning and/or memory disorders.
(6) A cognitive function improving agent comprising the composition of any of (1) to (4).
(7) The agent of (6), for improving a decline in learning and/or memory functions or learning and/or memory disorders.
(8) A food for improving cognitive function, comprising the composition of any of (1) to (4).
(9) The food of (8), for improving a decline in learning and/or memory functions or learning and/or memory disorders.
(10) A method for improving cognitive function of a subject in need of improving cognitive function, comprising allowing the subject to ingest a composition comprising glycine and serine, and having a content ratio of glycine to serine (glycine/serine) of not less than 0.6 in weight ratio, in an amount effective for improving cognitive function of the subject, or administering said amount of the composition to the subject.
(11) A method for improving cognitive function of a subject in need of improving cognitive function, comprising allowing the subject to ingest a composition comprising glycine and serine, and having a content ratio of glycine to serine (glycine/serine) of not less than 2 in weight ratio, in an amount effective for improving cognitive function of the subject, or administering said amount of the composition to the subject.
(12) The method of (10), wherein the composition further comprises one or more kinds selected from the group consisting of phospholipid, glutamic acid, cysteine, and cystine.
(13) The method of (11), wherein the composition further comprises one or more kinds selected from the group consisting of phospholipid, glutamic acid, cysteine, and cystine.
(14) The method of any of (10) to (13), which is a method for improving a decline in learning and/or memory functions or learning and/or memory disorders.
(15) A composition for improving or enhancing neuroprotective function of the hippocampus, comprising glycine and serine and having a content ratio of glycine to serine (glycine/serine) of not less than 0.6 in weight ratio.
(16) A composition for improving or enhancing neuroprotective function of the hippocampus, comprising glycine and serine and having a content ratio of glycine to serine (glycine/serine) of not less than 2 in weight ratio.
(17) The composition of (15), further comprising one or more kinds selected from the group consisting of phospholipid, glutamic acid, cysteine, and cystine.
(18) The composition of (16), further comprising one or more kinds selected from the group consisting of phospholipid, glutamic acid, cysteine, and cystine.

### Advantageous Effects of Invention

The present invention provides a composition for improving cognitive function that can effectively prevent or improve a decline in cognitive function and cognitive dysfunction.

The composition for improving cognitive function of the present invention is particularly effective for the prevention or improvement of a decline in learning and/or memory functions and learning and/or memory disorders.

In addition, the composition for improving cognitive function of the present invention is highly safe and suitable for continuous ingestion or administration.

Furthermore, the composition for improving cognitive function of the present invention can increase the expression of multiple gene groups involved in neuroprotection of the hippocampus in addition to the memory function of the hippocampus, and thus can also act as a composition for improving or enhancing the neuroprotective function of the hippocampus.

### BRIEF DESCRIPTION OF THE DRAWINGS

A more complete appreciation of the invention and many of the attendant advantages thereof will be readily obtained as the same become better understood by reference to the following detailed description when considered in connection with the accompanying drawings, wherein:

FIG. 1 is a diagram showing RI values during training trials and retention trials for each group of mice in Experimental Example 1.

FIG. 2 is a diagram showing RI values during training trials and retention trials for each group of mice in Experimental Example 2.

FIG. 3 is a diagram showing the results of clustering of the expression levels of the hippocampal genes of each group aligned by RNA sequencing in Experimental Example 3.

FIG. 4 is a diagram showing the expression levels (transcripts per million, TPM) of genes whose expression levels varied due to oral administration of the composition of Example 1 in Experimental Example 3. (A) shows the expression level of Sonic hedgehog (Shh) gene, (B) shows the expression level of Thyroid stimulating hormone receptor (TShr) gene, (C) shows the expression level of Family with sequence similarity 107 member a (Fam107a) gene, (D) shows the expression level of Galanin and GMAP prepropeptide (Gal) gene, (E) shows the expression level of D-site of albumin promoter (albumin D-box) binding protein (Dbp) gene.

### DETAILED DESCRIPTION OF THE PREFERRED EMBODIMENTS

The present invention provides a composition for improving cognitive function (hereinafter to be also referred to as “the composition of the present invention” in the present specification).

The composition of the present invention contains glycine and serin as effective components, and the content ratio of glycine to serine (glycine/serine) in the composition of the present invention is not less than 0.6 in weight ratio.

As used herein, the “cognitive function” in the present specification refers to higher-order functions of the brain, such as memory, judgment, calculation, comprehension, learning, thinking, language, performance, attention, orientation, execution, delayed recall and the like, and “improvement of cognitive function” refers to prevention or improvement of a decline in such cognitive function or disorder of such cognitive function.

The decline in the cognitive function and cognitive dysfunction that may be improved by the composition of the present invention includes a decline in cognitive function and cognitive dysfunction due to dementia caused by various diseases and lesions such as Alzheimer's disease, frontotemporal lobar degeneration (Pick disease etc.), Lewy body disease, cerebrovascular diseases and the like, a decline in cognitive function due to aging, a decline in cognitive function observed in healthy humans, for example, memory decline, reduction in attention, a decline in thinking skills and the like.

As described below, the composition of the present invention is particularly effective for the prevention or improvement of a decline in learning and/or memory functions and learning and/or memory disorders.

Glycine (2-aminoacetic acid) and serine (2-amino-3-hydroxypropanoic acid) contained in the composition of the present invention as effective components are non-essential amino acids contained in the tissues of various animals and plants.

The “serine” to be used may be any of an L form, a D form and a DL form. An L form and a DL form are preferably used, and an L form is more preferably used.

The “glycine” and “serine” can be used not only in a free form but also a salt form. The term “glycine” and “serine” in the present specification are concepts each encompassing even a salt. The salt form is not particularly limited as long as it is a pharmacologically acceptable or edible salt, and acid addition salt, salt with base and the like can be mentioned.

Concrete examples include salts with inorganic bases, salts with organic bases, salts with inorganic acids, salts with organic acids, salts with amino acid and the like.

Examples of the salts with inorganic bases include salts with alkali metals such as lithium, sodium, potassium and the like, salts with alkaline earth metals such as magnesium, calcium and the like, ammonium salt and the like.

Examples of the salts with organic bases include salts with alkanolamine such as monoethanolamine, diethanolamine, triethanolamine and the like, salts with heterocyclic amine such as morpholine, piperidine and the like, and the like.

Examples of the salts with inorganic acids include salts with hydrohalic acid (hydrochloric acid, hydrobromic acid, hydroiodic acid etc.), sulfuric acid, nitric acid, phosphoric acid and the like.

Examples of the salts with organic acids include salts with monocarboxylic acid such as formic acid, acetic acid, propanoic acid and the like; salts with saturated dicarboxylic acid such as oxalic acid, malonic acid, malic acid, succinic acid and the like; salts with unsaturated dicarboxylic acid such as maleic acid, fumaric acid and the like; salts with tricarboxylic acid such as citric acid and the like; salts with keto acid such as α-ketoglutaric acid and the like.

Examples of the salts with amino acid include salts with aliphatic amino acid such as alanine and the like; salts with aromatic amino acid such as tyrosine and the like; salts with basic amino acid such as arginine and the like; salts with acidic amino acid such as aspartic acid, glutamic acid and the like; salts with amino acid forming lactam such as pyroglutamic acid and the like.

Each of the above-mentioned salts may be a hydrate (salt hydrate), and examples of the hydrate include 1 hydrate to 6 hydrate and the like.

In the present invention, one kind each of “glycine” and “serine” in the above-mentioned free form or salt form may be used alone, or two or more kinds thereof may be used in combination.

For the object of the present invention, a free form of each of “glycine” and “serine” are preferable.

In the present invention, “glycine” and “serine” in a free form or salt form to be used may be extracted from animals, plants or the like, which are naturally present, and purified, or obtained by a chemical synthesis method, a fermentation method, an enzyme method or a gene recombinant method and the like. Commercially available products provided by each company may also be utilized.

In the composition of the present invention, a content ratio of glycine to serine (glycine/serine) is not less than 0.6 in weight ratio.

From the aspects of the effects of the present invention, a content ratio of glycine to serine (glycine/serine) in the composition of the present invention is preferably not less than 0.7, not less than 0.8, not less than 0.9, not less than 1, not less than 1.1, not less than 1.2, not less than 1.3, not less than 1.4, not less than 1.5, not less than 1.6, not less than 1.7, not less than 1.8, or not less than 1.9, more preferably not less than 2, each in weight ratio. A composition with the aforementioned content ratio of not less than 2 affords a good cognitive function-improving effect, as described below.

In addition, the composition of the present invention needs to contain serine, and a content ratio of glycine to serine (glycine/serine) in the composition of the present invention is preferably not more than 50 in weight ratio.

In the composition of the present invention, when both or either of glycine and serine are/is contained in a salt form, the content ratio of glycine to serine (glycine/serine) is calculated from the contents obtained by converting each content of glycine in a salt form and serine in a salt form to respective free forms.

The composition of the present invention may preferably further contain, in addition to glycine and serine at the above-mentioned content ratio, one or more kinds selected from the group consisting of phospholipid, glutamic acid, cysteine, and cystine.

By containing one or more kinds selected from the group consisting of phospholipid, glutamic acid, cysteine, and cystine in addition to glycine and serine at the above-mentioned content ratio, the cognitive function-improving effect can be enhanced.

Preferred phospholipids that can be contained in the composition of the present invention include glycerophospholipids such as phosphatidyl choline (lecithin), phosphatidyl serine, phosphatidyl ethanolamine, phosphatidyl inositol, phosphatidyl glycerol, and the like.

Phosphatidyl choline (lecithin) can be a source of choline in the biosynthetic pathway of the neurotransmission substance acetylcholine.

Phosphatidyl serine is a phospholipid that generally exists in the inner leaflet of cell membrane, and is also produced by base-exchange reaction of phosphatidyl ethanolamine. It is abundantly contained in the human brain and neural tissue, and has been reported to have an effect of improving brain function.

It is known that phosphatidyl inositol serves as a substrate for PI3 kinase (phosphoinositide 3-kinase) and acts as a second messenger in signal transduction.

Phosphatidyl glycerol is contained in large amounts in plant leaf and the like.

In the present invention, the above-mentioned phospholipid to be used may be extracted from animals, plants or the like, which are naturally present, and purified, or obtained by a chemical synthesis method, a fermentation method, an enzyme method or a gene recombinant method and the like. Commercially available products provided by each company may also be utilized.

In the present invention, one kind each of the above-mentioned phospholipids such as glycerophospholipid and the like may be used alone, or two or more kinds thereof may be used in combination.

For the purpose of the present invention, phosphatidyl choline (lecithin) and phosphatidyl serine are more preferably used.

Glutamic acid (2-aminopentanedioic acid) and cysteine (2-amino-3-mercaptopropanoic acid), which may be contained in the composition of the present invention, are amino acids that constitute glutathione together with glycine, and have an action of reducing neuroinflammation caused by oxidative stress, by synthesizing glutathione in the living body.

In addition, cystine (3,3′-dithio bis (2-aminopropanoic acid)) is an amino acid produced when two cysteine molecules bind via a disulfide bond (S—S), and functions as a source of cysteine because the disulfide bond is easily cleaved in the body.

In the present invention, glutamic acid, cysteine, and cystine to be used may be any of L-form, D-form, and DL-form. L-form and DL-form are preferably used, and L-form is more preferably used.

Furthermore, glutamic acid, cysteine, and cystine can be used not only in a free form but also in a salt form as described above for glycine and serine. Therefore, in the present invention, “glutamic acid”, “cysteine”, and “cystine” are concepts each also encompassing a salt.

In the composition of present invention, one kind each of “glutamic acid”, “cysteine”, and “cystine” in the above-mentioned free form or salt form may be used alone, or two or more kinds thereof may be used in combination.

For the purpose of the present invention, free forms of “glutamic acid”, “cysteine”, and “cystine” are preferably used.

In the present invention, glutamic acid, cysteine and cystine to be used may be extracted from animals, plants or the like, which are naturally present, and purified, or obtained by a chemical synthesis method, a fermentation method, an enzyme method or a gene recombinant method and the like. Commercially available products provided by each company may also be utilized.

In the composition of the present invention, glycine and serine, and one or more kinds selected from the group consisting of phospholipid, glutamic acid, cysteine, and cystine are preferably contained such that the content ratio of these [total content of glycine and serine:total content of one or more kinds selected from the group consisting of phospholipid, glutamic acid, cysteine, and cystine] is 10:1 to 1:10, more preferably 5:1 to 1:5, in weight ratio.

When one or more of glycine, serine, glutamic acid, cysteine, and cystine is/are contained in a salt form, the above-mentioned ratio is calculated from the contents obtained by converting the content of amino acid in the salt form to a free form.

The composition of the present invention may further contain, in addition to glycine and serine, or glycine and serine, and one or more kinds selected from the group consisting of phospholipid, glutamic acid, cysteine, and cystine, other nutrition components such as other amino acids, carbohydrates, lipids other than phospholipids, proteins, vitamins, minerals, and the like.

The composition of the present invention can be formulated into various forms such as liquids (e.g., solution, suspension, emulsion and the like); semi-solid (e.g., gel, cream and the like); solid (e.g., powder, granule, tablet, capsule and the like), and the like by adding, where necessary, other nutrition components, pharmaceutically acceptable additives, edible additives, and the like to glycine and serine, or glycine and serine and one or more kinds selected from the group consisting of phospholipid, glutamic acid, cysteine, and cystine, according to a formulating means well known in the field of preparations, for example, the methods described in the Japanese Pharmacopoeia XVII General Rules for preparations [3] Monographs for Preparations, which is incorporated herein by reference in its entirety, and the like.

The above-mentioned pharmaceutically acceptable additive or edible additive can be appropriately selected according to the form of the composition of the present invention and, for example, excipient, binder, disintegrant, lubricant, coating agent, base, solvent, solubilizing agents, solubilizer, emulsifier, dispersing agent, suspending agent, stabilizer, thickener, soothing agent, isotonicity agent, pH adjuster, antioxidant, antiseptic, preservative, corrigent, sweetener, flavor, colorant and the like can be mentioned.

To be specific, examples of the excipient include magnesium carbonate, saccharides (glucose, lactose, cornstarch etc.), sugar alcohol (sorbitol, mannitol etc.) and the like.

Examples of the binder include gelatin, pregelatinized starch, partly pregelatinized starch, cellulose and a derivative thereof (crystalline cellulose, hydroxypropylcellulose etc.) and the like.

Examples of the disintegrant include crospovidone, povidone, crystalline cellulose and the like.

Examples of the lubricant include talc, magnesium stearate and the like.

Examples of the coating agent include methacrylic acid-methyl methacrylate copolymer, methacrylic acid-ethyl acrylate copolymer, methyl methacrylate-butyl methacrylate-dimethylaminoethyl methacrylate copolymer, ethyl acrylate-methyl methacrylate-trimethylammonioethylmethacrylate chloride copolymer and the like.

Examples of the base include animal and plant fats and oils (olive oil, cacao butter, beef tallow, sesame oil, hydrogenated oil, castor oil etc.), wax (Carnauba wax, beeswax etc.), polyethylene glycol and the like.

Examples of the solvent include purified water, water for injection, monovalent alcohol (ethanol etc.), polyhydric alcohol (glycerol etc.) and the like.

Examples of the solubilizing agent include propylene glycol, medium-chain triglyceride and the like.

Examples of the solubilizer, emulsifier, dispersing agent and suspending agent include surfactant and the like such as sorbitan fatty acid ester, glycerol fatty acid ester, polyoxyethylene sorbitan fatty acid ester (polysorbate 20, polysorbate 80 etc.), polyoxyethylene hydrogenated castor oil, sucrose fatty acid ester and the like.

Examples of the stabilizer include adipic acid, β-cyclodextrin, ethylenediamine, sodium edetate and the like.

Examples of the thickener include water-soluble polymer (sodium polyacrylate, carboxyvinyl polymer etc.), polysaccharides (sodium alginate, xanthan gum, tragacanth etc.) and the like.

Examples of the soothing agent include ethyl aminobenzoate, chlorobutanol, propylene glycol, benzyl alcohol and the like.

Examples of the isotonicity agent include potassium chloride, sodium chloride, sorbitol, saline and the like.

Examples of the pH adjuster include hydrochloric acid, sulfuric acid, acetic acid, citric acid, lactic acid, sodium hydroxide, potassium hydroxide and the like.

Examples of the antioxidant include dibutylhydroxytoluene (BHT), butylhydroxyanisole (BHA), dl-α-tocopherol, erythorbic acid and the like.

Examples of the antiseptic and preservative include paraben (methylparaben etc.), benzyl alcohol, sodium dehydroacetate, sorbic acid and the like.

Examples of the corrigent include ascorbic acid, erythritol, disodium 5′-inosinate and the like.

Examples of the sweetener include aspartame, licorice extract, saccharin and the like.

Examples of the flavor include 1-menthol, d-camphor, vanillin and the like.

Examples of the colorant include tar pigment (Food Color Red No. 2, Food Color Blue No. 1, Food Color yellow No. 4 etc.), inorganic pigment (red iron oxide, yellow iron oxide, black iron oxide etc.), natural dye (turmeric extract, β-carotene, sodium copper-chlorophyllin, etc.) and the like.

In the present invention, one or more kinds of the above-mentioned additives can be used.

The content of glycine and serine in the composition of the present invention is generally 0.1 wt % to 100 wt %, preferably 1 wt % to 100 wt %, more preferably 10 wt % to 100 wt %, in the total amount of these.

The content of one or more kinds selected from the group consisting of phospholipid, glutamic acid, cysteine, and cystine in the composition of the present invention is preferably not more than 50 wt %, more preferably not more than 25 wt %.

When one or more of glycine, serine, glutamic acid, cysteine, and cystine is/are contained in a salt form, the content of amino acid in the salt form is shown in the contents obtained by converting to each free form.

The daily ingestion amount or dose of the composition of the present invention is appropriately determined according to the sex, age of the subject to be applied to (hereinafter to be also referred to as the “application subject” in the present specification), conditions and degree of a decline in cognitive function and cognitive dysfunction observed in the application subject, the form of the composition of the present invention, the administration method and the like. When the application subject is a human adult, it is generally 0.5 g to 50 g, preferably 1 g to 20 g, more preferably 2 g to 10 g, further preferably 3 g to 8 g, with respect to the total amount of glycine and serine (in the case of salt form, calculated by converting to the free form).

The above-mentioned amount can be ingested or administered at once or in several portions (for example, 2 to 4 portions) per day.

In addition, the ingestion or dosing period of the composition of the present invention is also appropriately determined according to the condition and symptoms of the application subject, and the like. Considering that a decline in cognitive function and cognitive dysfunction occur along with aging and various diseases, intracerebral lesions such as cerebral atrophy and the like, and the like, and chronically progresses, continuous ingestion or administration for a long period of time is preferable to improve cognitive function.

The composition of the present invention can be formulated as a unit package form. In the present specification, the “unit package form” means a form of one or more units with a particular amount (e.g., ingestion amount or dose per one time etc.) as one unit is/are filled in one container or packed in a package. For example, a unit package form with ingestion amount or dose per one time as one unit is referred to as “unit package form for ingestion amount or dose per one time”. A container or package used for the unit package form can be appropriately selected according to the form and the like of the composition of the present invention. For example, paper container or bag, plastic container or bag, pouch, aluminum can, steel can, glass bottle, pet bottle, PTP (press through pack) package sheet and the like can be mentioned.

The application subject of the composition of the present invention includes, for example, mammals (e.g., human, monkey, mouse, rat, guinea pig, hamster, rabbit, cat, dog, bovine, horse, donkey, swine, sheep, etc.), birds (e.g., duck, chicken, goose, turkey, etc.) and the like.

When the composition of the present invention is applied to an application subject animal (hereinafter to be also simply referred to as “subject animal”) other than human, the ingestion amount or dose of the composition of the present invention can be appropriately set according to the kind, sex, body weight and the like of the subject animal.

The composition of the present invention has a cognitive function improving effect, and is effective for the prevention or improvement of various symptoms or disorders caused by a decline in cognitive function and cognitive dysfunction such as a decline in learning and/or memory functions, learning and/or memory disorders, and the like, and particularly effective for the prevention or improvement of a decline in learning and/or memory functions and learning and/or memory disorders.

Since the composition of the present invention contains amino acids, which are contained in foods and have abundant food experience, as active ingredients, it is highly safe, suitable for continuous ingestion or administration, and thus suitable for preventing or improving a decline in cognitive function and cognitive dysfunction that chronically progresses along with aging and the like.

Therefore, the composition of the present invention can be preferably ingested by or administered to those exhibiting symptoms or disorders due to a decline in cognitive function and cognitive dysfunction, elderly people and middle- or late middle-aged persons requiring improvement of cognitive function, and the like. The composition of the present invention can be particularly preferably ingested by or administered to those exhibiting a decline in learning and/or memory functions and learning and/or memory disorders, and elderly people and middle- or late middle-aged persons in need of improvement in a decline in learning and/or memory functions and learning and/or memory disorders, and the like.

As described below, the composition of the present invention has an action of increasing the expression of genes relating to cell proliferation of endothelial cell, fibroblast, mesenchymal cell, and the like, vascular vessel angiogenesis, differentiation of dopaminergic neuron, organizing intermediate filament cytoskeleton, regulation of growth factor signaling pathway and the like of fibroblast growth factor and the like, regulation of nerve cell death, regulation of active oxygen species metabolic pathway, and the like, and decreasing the expression of Gal gene encoding galanin, a type of neuropeptide, and Dbp gene relating to synapse plasticity of the hippocampus.

Dopaminergic neurons involving genes whose expression is increased by the composition of the present invention, are known to control a wide range of brain functions, including cognitive functions, and regulation of neuronal cell death relates to neuroprotective action. Furthermore, it has been reported that the Gal gene, whose expression is reduced by the composition of the present invention, inhibits cholinergic transmission in the hippocampus, and that overexpression of the Dbp gene reduces spatial cognitive function.

Therefore, it is suggested that the cognitive function-improving effect afforded by the composition of the present invention may be provided through the above-mentioned variation in gene expression.

In addition, since the composition of the present invention has the action of changing the above-mentioned gene expression, the composition of the present invention may be effective for maintenance of normal cells; continuance of functional operation; repair and regeneration of cell associated with aging, chemical or mechanical lesion; and prevention of degeneration and immaturity resulting from loss of differentiation under pathogenic conditions, such as encephalitis and meningitis due to infection with pathogenic microorganisms such as viruses, bacteria, fungi, protozoa, and parasites.

Specifically, it can be applied to the prevention or treatment of neurological symptoms caused by injury, damage, disease, and the like (cure, improvement, or alleviation of the symptoms). Injuries that cause neurological symptoms include (i) acute, subacute, or chronic injuries to the nervous system (e.g., wound injuries, chemical injuries, chemical damages, vascular damages and defects (ischemia caused by cerebrovascular disorders) (these accompany infectious or inflammatory, tumor inducible injuries); (ii) chronic neurodegenerative diseases of the nervous system (e.g. Parkinson's disease, Huntington's chorea, amyotrophic lateral sclerosis, etc. (such diseases include spinocerebellar degeneration); and (iii) chronic immune diseases of the nervous system or injured nervous system (e.g. multiple sclerosis, etc.).

Particularly, the gene groups whose expression is increased by the composition of the present invention, include multiple genes involved in neuroprotection of the hippocampus in addition to memory functions, as described below. Therefore, the composition of the present invention can show an action of improving or enhancing the neuroprotective function of the hippocampus.

Therefore, the composition of the present invention can also act as a composition for improving or enhancing neuroprotective function of the hippocampus.

The composition of the present invention can be provided as a cognitive function improving agent (hereinafter to be also referred to as “the agent of the present invention” in the present specification) directly or by further adding the above-mentioned pharmaceutically acceptable additives.

The agent of the present invention can have a dosage form of oral preparation such as tablet, coated tablet, chewable tablet, pill, (micro)capsule, granule, fine granule, powder, elixir, lemonade, syrup, suspension, emulsion, oral jelly and the like, injectable preparation such as solution, suspension, emulsion and the like for injection, solid injection to be used by dissolving or suspending when in use, transfusion, sustainable injection and the like, tubal liquid, and the like.

The agent of the present invention may contain an anti-dementia drug as long as the characteristics of the present invention are not impaired.

Examples of the anti-dementia drug include acetylcholinesterase inhibitors such as donepezil hydrochloride, galanthamine, rivastigmine and the like; and NMDA receptor antagonists such as memantine and the like, and these can be used according to general usage and dosage.

The agent of the present invention can be preferably administered to patients exhibiting symptoms or disorders due to decline in cognitive function and cognitive dysfunction such as a decline in learning and/or memory functions, learning and/or memory disorders, and the like, patients who may develop the aforementioned symptoms or disorders, elderly people and middle- or late middle-aged persons exhibiting a decline in cognitive function and cognitive dysfunction, and the like, and particularly preferably administered to those exhibiting a decline in learning and/or memory functions and learning and/or memory disorders, and elderly people and middle- or late middle-aged persons in need of improvement in a decline in learning and/or memory functions and learning and/or memory disorders, and the like.

The agent of the present invention is administered per day to the above-mentioned application subject such that the total amount of glycine and serine (in the case of salt form, calculated by converting to the free form) is the above-mentioned daily dose.

Furthermore, the composition of the present invention can be ingested by adding to various foods. The food to which the composition of the present invention is added is not particularly limited, and may be any as long as it is a food in the form generally served for meals or dessert.

For example, the composition of the present invention is added to drinks such as beverage water and the like, and a suitable flavor is added when desired, whereby a drink can be provided.

More specifically, the composition of the present invention can be added, for example, to beverage water such as fruit juice drinks, sport drinks and the like; dairy products such as milk, yogurt and the like; confectionery such as jelly, chocolate, candy, biscuit and the like, and the like.

The composition of the present invention is preferably added to the above-mentioned various foods in the amounts to be ingested per day such that the total amount of glycine and serine (in the case of salt form, calculated by converting to the free form) is the above-mentioned daily ingestion amount.

The composition of the present invention can be provided as a food for improving cognitive function (hereinafter to be also referred to as “the food of the present invention” in the present specification) directly or by adding general food additives as necessary and according to a general food production technique.

The food of the present invention can be prepared as various forms such as liquid (e.g., solution, suspension, emulsified liquid and the like); semi-solid (e.g., gel, cream and the like); solid (e.g., powder, granule, sheet, capsule, tablet and the like), and the like.

Furthermore, the food of the present invention can be prepared as various food forms such as beverage water (fruit juice drinks, sport drinks, coffee drinks, tea drinks etc.), dairy product (lactic fermenting beverage, fermented milk, butter, cheese, yogurt, processed milk, defatted milk etc.), meat product (ham, sausage, hamburger etc.), fish meat processed seafood paste product (fish cake, tube-shaped fish sausage, deep-fried ball of fish paste etc.), egg product (rolled Japanese-style egg omelette, steamed egg custard etc.), confectionery (cookie, jelly, chewing gum, candy, snack food, frozen dessert etc.), bread, noodles, pickle, dried fish, food boiled in soy sauce, soup, seasoning and the like by adding the composition of the present invention to various food starting materials and adding general food additives as necessary. It may also be a bottled food, canned food or retort pouch food.

As the above-mentioned food additive, manufacturing agent (brine, binding agent, etc.), thickening stabilizer (xanthan gum, sodium carboxymethylcellulose, etc.), gelation agent (gelatin, agar, carrageenan, etc.), gum base (vinyl acetate resin, jelutong, chicle, etc.), emulsifier (glycerol fatty acid ester, sucrose fatty acid ester, sorbitan fatty acid ester, saponin, etc.), preservative (benzoic acid, sodium benzoate, sorbic acid, potassium sorbate, ε-polylysine, etc.), antioxidant (ascorbic acid, erythorbic acid, catechin, etc.), glazing agent (shellac, paraffin wax, beeswax, etc.), fungicide (thiabendazole, fludioxonil, etc.), leavening agent (sodium hydrogen carbonate, glucono-δ-lactone, alum, etc.), sweetener (aspartame, acesulfame potassium, licorice extract, etc.), bittering agent (caffeine, naringin, worm wood extract, etc.), acidulant (citric acid, tartaric acid, lactic acid, etc.), seasoning (sodium L-aspartate, disodium 5′-inosinate, etc.), colorant (annatto dye, turmeric dye, gardenia dye, etc.), flavor (synthetic flavor such as ethyl acetoacetate, anisaldehyde, and the like, natural flavor such as orange, lavender, and the like) and the like can be mentioned.

In the present invention, one or more kinds of the above-mentioned food additives can be used.

The food of the present invention can be preferably ingested by elderly people and middle- or late middle-aged persons showing a decline in cognitive functions and cognitive dysfunctions such as a decline in learning and/or memory functions, learning and/or memory disorders, and the like, elderly people and middle- or late middle-aged persons at a risk of showing a decline in cognitive function and cognitive dysfunction, and further, a wide range of subjects for the purpose of preventing a decline in cognitive function, and particularly preferably ingested by elderly people and middle- or late middle-aged persons showing a decline in learning and/or memory functions and learning and/or memory disorders, elderly people and middle- or late middle-aged persons at a risk of showing a decline in learning and/or memory functions and learning and/or memory disorders, and further, a wide range of subjects for the purpose of preventing a decline in learning and/or memory functions.

Therefore, the food of the present invention can also be provided as food with health claims such as food for specified health uses, food with nutrient function claims, indicated functional food and the like, special purpose foods such as food for sick people, food for the elderly and the like, health supplement and the like for improving cognitive function.

The food of the present invention is preferably ingested per day by the above-mentioned application subject such that the total amount of glycine and serine (in the case of salt form, calculated by converting to the free form) is the above-mentioned daily ingestion amount.

Furthermore, the present invention also provides a method for improving cognitive function in a subject animal in need of improvement of cognitive function (hereinafter to be also referred to as the “method of the present invention” in the present specification).

The method of the present invention includes allowing a subject animal in need of improving cognitive function to ingest a composition containing glycine and serine and having a content ratio of glycine to serine (glycine/serine) of not less than 0.6 in weight ratio, in an amount effective for improving cognitive function of the subject animal, or administering said amount of the composition to the animal.

In the method of the present invention, a content ratio of glycine to serine (glycine/serine) in the composition to be ingested by or administered to a subject animal is preferably not less than 0.7, not less than 0.8, not less than 0.9, not less than 1, not less than 1.1, not less than 1.2, not less than 1.3, not less than 1.4, not less than 1.5, not less than 1.6, not less than 1.7, not less than 1.8, or not less than 1.9, more preferably not less than 2, each in weight ratio. Ingestion or administration of a composition with the aforementioned content ratio of not less than 2 affords a good improving effect on the cognitive function of the subject animal, as described below.

In the method of the present invention, the composition to be ingested by or administered to a subject animal needs to contain serine, and a content ratio of glycine to serine (glycine/serine) in the composition of the present invention is preferably not more than 50 in weight ratio.

In the method of the present invention, it is preferable to allow a subject animal in need of improving cognitive function to ingest a composition containing glycine and serine at the above-mentioned content ratio, as well as one or more kinds selected from the group consisting of phospholipid, glutamic acid, cysteine, and cystine, or administer the composition to the subject animal.

In the composition to be ingested by or administered to a subject animal in the method the present invention, glycine and serine, and one or more kinds selected from the group consisting of phospholipid, glutamic acid, cysteine, and cystine are preferably contained such that the content ratio of these [total content of glycine and serine:total content of one or more kinds selected from the group consisting of phospholipid, glutamic acid, cysteine, and cystine] is 10:1 to 1:10, more preferably 5:1 to 1:5, in weight ratio.

The content of glycine and serine in the composition to be ingested by or administered to a subject animal in need of improvement in the cognitive function in the method of the present invention is generally 0.1 wt % to 100 wt %, preferably 1 wt % to 100 wt %, more preferably 10 wt % to 100 wt %, in the total amount of these.

The content of one or more kinds selected from the group consisting of phospholipid, glutamic acid, cysteine, and cystine in the composition to be ingested by or administered to a subject animal in need of improvement in the cognitive function in the method of the present invention is preferably not more than 50 wt %, more preferably not more than 25 wt %.

When one or more of glycine, serine, glutamic acid, cysteine, and cystine is/are contained in a salt form, a content ratio of glycine to serine, a content ratio of glycine and serine, and one or more kinds selected from the group consisting of phospholipid, glutamic acid, cysteine, and cystine, and each content of the aforementioned amino acids are calculated or shown with the contents obtained by converting the content of amino acid in a salt form to that in a free form.

The subject animal in the method of the present invention includes mammal (e.g., human, monkey, mouse, rat, guinea pig, hamster, rabbit, cat, dog, bovine, horse, donkey, swine, sheep etc.), birds (e.g., duck, chicken, goose, turkey etc.) and the like.

The effective amount of the composition containing glycine and serine at the above-mentioned content ratio in the method of the present invention is appropriately determined according to the kind, age, sex, the condition or level of a decline in cognitive function and cognitive dysfunction, and the like of the subject animal. An amount similar to the above-mentioned ingestion amount or dose of the composition of the present invention for a human or a subject animal other than human can be ingested or administered at the frequency and period mentioned above.

For example, when the subject animal is a human adult, the above-mentioned effective amount is generally 0.5 g to 50 g, preferably 1 g to 20 g, more preferably 2 g to 10 g, further preferably 3 g to 8 g, with respect to the total amount of glycine and serine (in the case of salt form, calculated by converting to the free form), and can be ingested or administered at once or in several portions (e.g., 2 to 4 portions) per day.

As an ingestion or administration method of the composition containing glycine and serine at the above-mentioned content ratio in the method of the present invention, oral ingestion or oral administration, enteral tube administration, administration by infusion and the like can be mentioned. Oral ingestion or oral administration is preferable since convenient ingestion is possible without the need to perform under the guidance and supervision of a doctor at a medical institution.

The method of the present invention is effective for the prevention or improvement of various symptoms or disorders caused by a decline in cognitive function and cognitive dysfunction such as a decline in learning and/or memory functions, learning and/or memory disorders, and the like.

In the case of human, the method of the present invention is preferably applied to patients exhibiting symptoms and disorders due to a decline in cognitive function and cognitive dysfunction such as a decline in learning and/or memory functions, learning and/or memory disorders and the like, and elderly people and middle- or late middle-aged persons requiring prevention of a decline in cognitive function such as elderly people and middle- or late middle-aged persons at a risk of showing a decline in cognitive function and cognitive dysfunction, and particularly preferably applied to those exhibiting a decline in learning and/or memory functions and learning and/or memory disorders, and elderly people and middle- or late middle-aged persons in need of improvement in a decline in learning and/or memory functions and learning and/or memory disorders.

Particularly, since the method of the present invention uses amino acids, which are contained in foods and have abundant food experience, as an active ingredient, it has high safety and can be applied continuously.

Other features of the invention will become apparent in the course of the following descriptions of exemplary embodiments which are given for illustration of the invention and are not intended to be limiting thereof.

### EXAMPLES

### Example 1. Composition for Improving Cognitive Function

To afford the composition shown in Table 1, respective components were weighed and mixed to prepare the composition for improving cognitive function of Example 1 (hereinafter to be referred to as “the composition of Example 1”). Similarly, as a comparison sample, a composition with the composition ratio shown in Table 1 was prepared and used as the composition of Comparative Example 1, and glycine (3 g) was used as the agent of Comparative Example 2.

TABLE 1

| | content (g) | | |
|---|---|---|---|
| | | Comparative | Comparative |
| component | Example 1 | Example 1 | Example 2 |
| glycine | 2 | 1 | 3 |
| L-serine | 1 | 2 | 0 |
| total amount | 3 | 3 | 3 |

### Experimental Example 1. Study of Action of Compositions of Example 1 and Comparative Example 1, and Agent of Comparative Example 2 on Learning and/or Memory Functions

Using C57Bl/6j mice (12-week-old, male) (purchased from CHARLES RIVER LABORATORIES JAPAN, INC.), a lipopolysaccharide (LPS)-induced learning and/or memory disorder model was prepared, and an action of the compositions of Example 1 and Comparative Example 1 and the agent of Comparative Example 2 on learning and/or memory functions was studied.

C57Bl/6j mice were divided into 4 groups as shown in Table 2 (n=18/group).

To the control LPS group was orally administered 10 mL of distilled water per kg of mouse body weight once a day for 13 consecutive days.

The composition of Example 1 was suspended in distilled water at a concentration of 0.3 g/mL, and 10 mL (3 g/kg body weight) was orally administered per kg of mouse body weight once a day for 13 consecutive days to the group to be administered with the composition of Example 1 (LPS+Gly2/L-Ser1).

The composition of Comparative Example 1 was suspended in distilled water at a concentration of 0.3 g/mL, and 10 mL (3 g/kg body weight) was orally administered per kg of mouse body weight once a day for 13 consecutive days to the group to be administered with the composition of Comparative Example 1 (LPS+Gly1/L-Ser2).

The agent of Comparative Example 2 was suspended in distilled water at a concentration of 0.3 g/mL, and 10 mL (3 g/kg body weight) was orally administered per kg of mouse body weight once a day for 13 consecutive days to the group to be administered with the agent of Comparative Example 2 (LPS+Gly3).

In each of the above groups, an LPS solution (25 μg/mL) prepared by dissolving LPS in physiological saline was intraperitoneally administered at 10 mL per kg body weight (250 μg/kg body weight) from day 4 to day 10 of administration of distilled water, each composition of Example 1 and Comparative Example 1, and the agent of Comparative Example 2, at 30 min after each oral administration.

TABLE 2

| | administered substance | |
|---|---|---|
| | | intraperitoneal |
| group | oral administration | administration |
| LPS | distilled water | LPS |
| LPS + Gly1/L-Ser2 | suspension containing | LPS |
| | composition of Comparative | |
| | Example 1 | |
| LPS + Gly2/L-Ser1 | suspension containing | LPS |
| | composition of Example 1 | |
| LPS + Gly3 | suspension containing agent | LPS |
| | of Comparative Example 2 | |

As a system for evaluating the learning and/or memory functions of mice, a novel object recognition test was performed as described below.
(i) Mice were moved from their home cages to a plastic experimental arena surrounded by a wall (40 cm long×40 cm wide and 40 cm high), and allowed to move freely for 12 min for acclimation (acclimation trial).
(ii) After 24 hr from the acclimation trial, the mice were transferred to an experimental arena with the same objects A1 and A2 placed at predetermined positions, and their behaviors were video recorded for 10 min (training trial).
(iii) After 24 hr from the training trial, object A1 or A2 placed in the experimental arena and having a shorter contact time in the training trial was replaced with a new object B, the mice were placed in the aforementioned experimental arena again, and the behavior was video recorded for 10 min (retention trial).
(iv) Using the recorded videos, the contact time of the mice to the object during training trial and retention trial was measured.
(v) Recognition Index (RI) was determined as follows.

During the training trial, the ratio (%) of the time of contact with object A1 or A2 with a shorter contact time to the total time of contact with the two objects (A1 and A2) placed in the experimental arena was calculated.

During the retention trial, the ratio (%) of the time of contact with object B to the total time of contact with the two objects (A1 and B, or A2 and B) placed in the experimental arena was determined. A higher RI value during the retention trial indicates that the mice remembered the object.

In addition, individuals whose contact with the same two objects was biased during the training trial (the ratio of the time of contact with object A1 or A2 with shorter contact time to the total contact time was less than 30%) were excluded from the analysis. Individuals whose total time of contact with the two objects during the training trial and retention trial was less than 10 seconds were also excluded from the analysis.

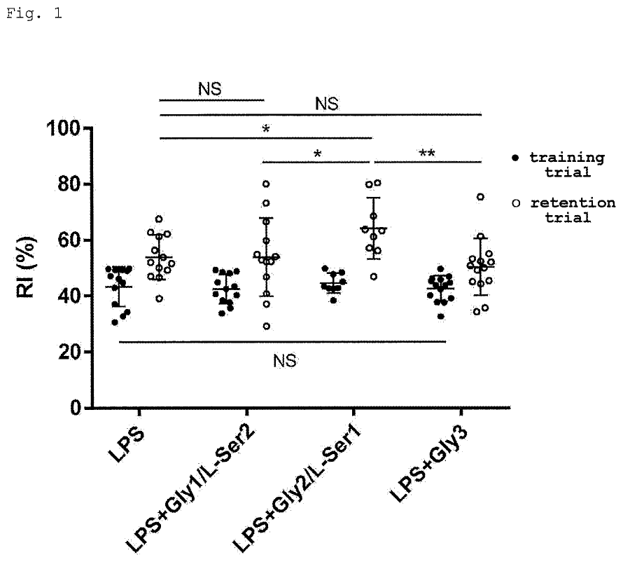

For each group, an acclimation trial was performed after the administration of distilled water, each composition of Example 1 and Comparative Example 1, and the agent of Comparative Example 2 on day 11, a training trial was performed after the administration on day 12, and a training trial was performed after the administration on day 13. The RI values (%) for the mice in each group during training trial and retention trial are shown in FIG. 1. In the Figure, the mean RI value of each group is shown by a horizontal line, and the standard deviation is shown by an error bar.

A repeated two-way analysis of variance was performed on the RI values (%) during training trial and retention trial for all groups, and Tukey's multiple comparison test was performed for comparison between groups during training trial and retention trial. In the Figure, “*” indicates that there is a significant difference between the compared groups at P<0.05, and “**” indicates that there is a significant difference between the compared groups at P<0.01. Moreover, “NS” indicates that no significant difference is observed between the compared groups.

As shown in FIG. 1, no significant difference was observed between the groups in the RI value (%) during the training trial.

On the other hand, regarding the RI value (%) during the retention trial, the RI value (%) was significantly higher (p<0.05 for LPS and LPS+Gly1/L-Ser2, P<0.01 for LPS+Gly3) in the group orally administered with the composition of Example 1 of the present invention (LPS+Gly2/L-Ser1), as compared with the group orally administered with distilled water (LPS), the group orally administered with the composition of Comparative Example 1 (LPS+Gly1/L-Ser2), and the group orally administered with the agent of Comparative Example 2 (LPS+Gly3). However, a significant difference was not observed in the RI values (%) of the groups respectively orally administered with the composition of Comparative Example 1 and the agent of Comparative Example 2 (LPS+Gly1/L-Ser2 and LPS+Gly3), as compared with the RI value (%) of the group orally administered with distilled water (LPS).

### Example 2. Composition for Improving Cognitive Function

To afford the composition shown in Table 3, respective components were weighed and mixed to prepare a composition for improving cognitive function of Example 2 (hereinafter sometimes to be referred to as “the composition of Example 2”). Similarly, a composition with the formulation shown in Table 3 was prepared and used as the composition of Comparative Example 3.

TABLE 3

| | content (g) | |
|---|---|---|
| component | Example 2 | Comparative Example 3 |
| glycine | 2 | 1 |
| L-serine | 1 | 2 |
| L-cystine | 0.5 | 0.5 |
| soybean lecithin | 0.5 | 0.5 |
| total amount | 4 | 4 |

### Experimental Example 2. Study of Action of Compositions of Example 2 and Comparative Example 3 on Learning and/or Memory Functions

In the same manner as in Experimental Example 1, using C57Bl/6j mice (12-week-old, male) (purchased from CHARLES RIVER LABORATORIES JAPAN, INC.), a lipopolysaccharide (LPS)-induced learning and/or memory disorder model was prepared, and an action of the compositions of Example 2 and Comparative Example 3 on learning and/or memory functions was studied.

C57Bl/6j mice were divided into 3 groups as shown in Table 4 (n=18/group).

To the control LPS group was orally administered 10 mL of 0.5 wt % methylcellulose aqueous solution per kg of mouse body weight once a day for 13 consecutive days.

The composition of Example 2 was suspended in 0.5 wt % methylcellulose aqueous solution at a concentration of 0.4 g/mL, and 10 mL (4 g/kg body weight) was orally administered per kg of mouse body weight once a day for 13 consecutive days to the group to be administered with the composition of Example 2 (LPS+Gly/Ser mix1).

The composition of Comparative Example 3 was suspended in 0.5 wt % methylcellulose aqueous solution at a concentration of 0.4 g/mL, and 10 mL (4 g/kg body weight) was orally administered per kg of mouse body weight once a day for 13 consecutive days to the group to be administered with the composition of Comparative Example 3 (LPS+Gly/Ser mix2).

In each of the above groups, an LPS solution (25 μg/mL) prepared by dissolving LPS in physiological saline was intraperitoneally administered at 10 mL per kg body weight (250 μg/kg body weight) from day 4 to day 10 of administration of 0.5 wt % methylcellulose aqueous solution and each composition of Example 2 and Comparative Example 3, at 30 min after each oral administration.

TABLE 4

| | administered substance | |
|---|---|---|
| | | intraperitoneal |
| group | oral administration | administration |
| LPS | solvent (0.5% | LPS |
| | methylcellulose aqueous | |
| | solution) | |
| LPS + Gly/Ser | suspension containing | LPS |
| mix1 | composition of Example 2 | |
| LPS + Gly/Ser | suspension containing | LPS |
| mix2 | composition of Comparative | |
| | Example 3 | |

The actions of the compositions of Example 2 and Comparative Example 3 on learning and/or memory functions were evaluated by performing a novel object recognition test in the same manner as in Experimental Example 1.

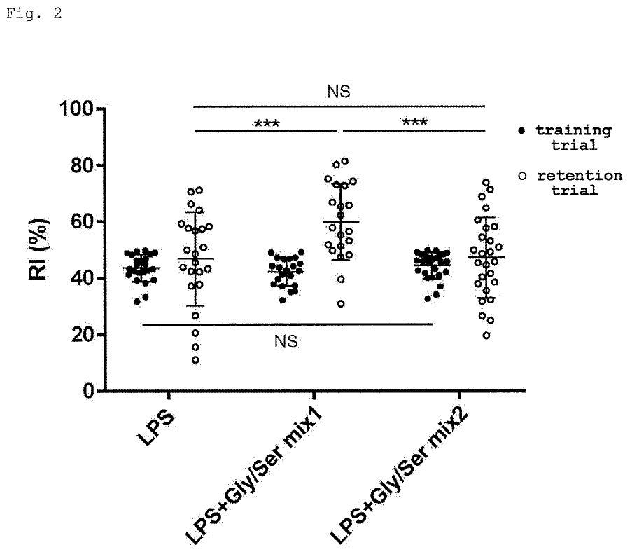

An acclimation trial was performed after the administration of the compositions of Example 2 and Comparative Example 3 on day 11, a training trial was performed after the administration on day 12, and a training trial was performed after the administration on day 13. The RI values (%) for the mice in each group during training trial and retention trial were calculated and are shown in FIG. 2. In the Figure, the mean RI value of each group is shown by a horizontal line, and the standard deviation is shown by an error bar.

A repeated two-way analysis of variance was performed on the RI values (%) during training trial and retention trial for all groups, and Tukey's multiple comparison test was performed for comparison between groups during training trial and retention trial. In the Figure, “***” indicates that there is a significant difference between the compared groups at P<0.001. Moreover, “NS” indicates that no significant difference is observed between the compared groups.

As shown in FIG. 2, no significant difference was observed between the groups in the RI value (%) during the training trial.

On the other hand, regarding the RI value (%) during the retention trial, the RI value (%) was significantly higher in the group orally administered with the composition of Example 2 of the present invention (LPS+Gly/Ser mix1), as compared with each of the group orally administered with a solvent alone (LPS) and the group orally administered with the composition of Comparative Example 3 (LPS+Gly/Ser mix2) (p<0.001 for each of LPS and LPS+Gly/Ser mix2). However, a significant difference was not observed in the RI value (%) of the group orally administered with the composition of Comparative Example 3 (LPS+Gly/Ser mix2), as compared with each of the group orally administered with a solvent alone (LPS).

From the above-mentioned results of Experimental Example 1, it was suggested that the composition of Example 1 of the present invention improves learning and/or memory functions and improves decreased learning and/or memory functions in a learning and/or memory disorder mouse model induced by intraperitoneal administration of LPS.

From the above-mentioned results of Experimental Example 2, it was suggested that a composition obtained by adding cystine and lecithin to the composition of Example 1 similarly improves a decline in learning and/or memory functions.

### Experimental Example 3. Study of Action of Composition of Example 1 on Gene Expression of Mouse Hippocampus

In the same manner as in Experimental Example 1, using C57Bl/6j mice (12-week-old, male) (purchased from CHARLES RIVER LABORATORIES JAPAN, INC.), a lipopolysaccharide (LPS)-induced learning and/or memory disorder model was prepared, and an action of composition of Example 1 on gene expression of mouse hippocampus was studied. At that time, as comparison samples, 2 g of glycine was used as the agent of Comparative Example 4, and 1 g of L-serine was used as the agent of Comparative Example 5, and the action on gene expression in the mouse hippocampus was similarly examined.

C57Bl/6j mice were divided into 4 groups as shown in Table 5 (n=5/group).

To the control LPS group was orally administered 10 mL of 0.5 wt % methylcellulose aqueous solution per kg of mouse body weight once a day for 13 consecutive days.

The composition of Example 1 was suspended in 0.5 wt % methylcellulose aqueous solution at a concentration of 0.3 g/mL, and 10 mL (3 g/kg body weight) was orally administered per kg of mouse body weight once a day for 13 consecutive days to the group to be administered with the composition of Example 1 (LPS+Gly2/L-Ser1).

The agent of Comparative Example 4 was suspended in 0.5 wt % methylcellulose aqueous solution at a concentration of 0.2 g/mL, and 10 mL (2 g/kg body weight) was orally administered per kg of mouse body weight once a day for 13 consecutive days to the group to be administered with the agent of Comparative Example 4 (LPS+Gly2).

The agent of Comparative Example 5 was suspended in 0.5 wt % methylcellulose aqueous solution at a concentration of 0.1 g/mL, and 10 mL (1 g/kg body weight) was orally administered per kg of mouse body weight once a day for 13 consecutive days to the group to be administered with the agent of Comparative Example 5 (LPS+L-Ser1).

In each of the above groups, an LPS solution (25 μg/mL) prepared by dissolving LPS in physiological saline was intraperitoneally administered at 10 mL per kg body weight (250 μg/kg body weight) from day 4 to day 10 of administration of 0.5 wt % methylcellulose aqueous solution, the composition of Example 1 and respective agents of Comparative Examples 4, 5, at 30 min after each oral administration.

TABLE 5

| | administered substance | |
|---|---|---|
| | | intraperitoneal |
| group | oral administration | administration |
| LPS | solvent (0.5% methylcellulose | LPS |
| | aqueous solution) | |
| LPS + Gly2/L- | suspension containing | LPS |
| Ser1 | composition of Example 1 | |
| LPS + Gly2 | suspension containing agent of | LPS |
| | Comparative Example 4 | |
| LPS + L-Ser1 | suspension containing agent of | LPS |
| | Comparative Example 5 | |

Hippocampal tissue was collected from each group of mice that had been administered with each suspension or vehicle shown in Table 5 for 13 consecutive days, total RNA was extracted from each individual separately, and RNA sequencing (RNA-seq) was performed. An average of 38 million paired-end reads were obtained per sample, and 92.21% were aligned to the mouse genome assembly (mm39). Samples from individuals suspected of being contaminated at the time of tissue collection were excluded from analysis.

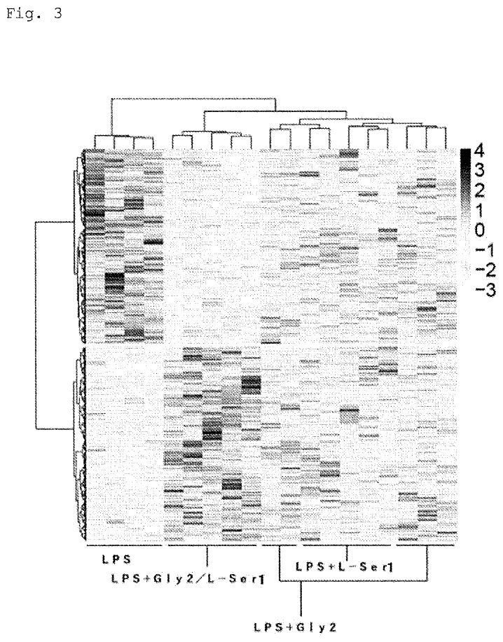

Using the package DESeq2 of the statistical analysis software R, a group orally administered with the composition of Example 1 of the present invention (LPS+Gly2/L-Ser1) and a group orally administered with a solvent alone (LPS) were compared, and genes that showed varied expression were extracted. Distances were calculated using the Pearson correlation coefficient for the extracted gene groups, and clustering was performed using the Ward method. it was clarified that Hippocampus tissue obtained from a mouse of the group orally administered with the composition of Example 1 of the present invention (LPS+Gly2/L-Ser1) contains 346 up-regulated genes and 348 down-regulated genes as compared with the group orally administered with solvent alone (LPS) (FIG. 3).

The package clusterProfiler of the statistical analysis software R was used for the clustering processing. Then, gene ontology (GO) analysis was performed. It was clarified that the upregulated genes were involved in cell proliferation such as endothelial cells, fibroblasts, and mesenchymal cells, differentiation of dopaminergic neurons, organization of the intermediate filament cytoskeleton, and regulation of growth factor signaling pathways such as fibroblast growth factor (Table 6).

TABLE 6

| Description | p.adjust | gene ID |
|---|---|---|
| negative regulation of cellular response to | 0.0001 | Vwc21/Fbn1/Agt/Ngfr/Wnt5a/Shisa2/Wnt1/Xdh/Ofd1 |
| growth factor stimulus | | |
| fibroblast growth factor receptor signaling | 0.0012 | Fgf23/Ccn2/Ngfr/Churc1/Wnt5a/Shisa2/Ofd1 |
| pathway | | |
| endothelium development | 0.0019 | Slc40a1/Sox18/Hapln2/Kdr/Prok2/Agt/Stc1/Xdh |
| response to fibroblast growth factor | 0.0019 | Fgf23/Ccn2/Ngfr/Churc1/Wnt5a/Shisa2/Ofd1 |
| endothelial cell differentiation | 0.0053 | Sox18/Hapln2/Kdr/Prok2/Agt/Stc1/Xdh |
| steroid metabolic process | 0.0057 | Hsd17b7/Rorc/Hsd3b6/Shh/Fgf23/Sult1a1/Msmo1/Agt/ |
| | | Pctp/Cyp1b1/Stard4 |
| endothelial cellular proliferation | 0.0090 | Kdr/Prok2/Hmox1/Vip/Ngfr/Thbs4/Wnt5a/Xdh |
| reactive oxygen species metabolic process | 0.0097 | Pdk4/Agt/Ccn2/Ngfr/Tfap2a/Cdkn1a/Xdh/Cyp1b1/Cybb |
| cellular response to iron ion | 0.0138 | Slc40a1/Hmox1/Tfap2a |
| regulation of epithelial cellular | 0.0155 | Shh/Kdr/Foxp2/Ceacam2/Hmox1/Vip/Ngfr/Thbs4/Wnt5a/ |
| proliferation | | Rida/Xdh |
| positive regulation of mesenchymal cellular | 0.0160 | Shh/Kdr/Foxp2/Wnt5a |
| proliferation | | |
| positive regulation of fibroblast | 0.0167 | Agt/Ngfr/Wnt5a/Wnt1/Cdkn1a |
| proliferation | | |
| dopaminergic neuron differentiation | 0.0185 | Shh/Tshr/Wnt5a/Wnt1 |
| chromosome condensation | 0.0294 | Ern2/Ncapg2/H1f2/H1f0 |
| regulation of neuron death | 0.0323 | Shh/Kdr/Hmox1/Mt1/Agt/Ccl3/Ngfr/Tfap2a/Wnt5a/Wnt1 |
| kidney development | 0.0399 | Fbn1/Shh/Agt/Cep290/Tfap2a/Wnt5a/Wnt1/Plce1/Amer1 |
| response to iron ion | 0.0399 | Slc40a1/Hmox1/Tfap2a |
| axis specification | 0.0399 | Shh/Mesp2/Wnt5a/Stc1/Wnt1 |
| intermediate filament cytoskeleton | 0.0399 | Pkp1/Flg/Shh/Prph |
| organization | | |
| vasculogenesis | 0.0399 | Sox18/Shh/Kdr/Prok2/Xdh |
| intermediate filament-based process | 0.0399 | Pkp1/Flg/Shh/Prph |
| estrogen metabolic process | 0.0399 | Hsd17b7/Sult1a1/Cyp1b1 |
| renal system development | 0.0399 | Fbn1/Shh/Agt/Cep290/Tfap2a/Wnt5a/Wnt1/Plce1/Amer1 |
| androgen metabolic process | 0.0399 | Hsd17b7/Hsd3b6/Shh |
| mesenchymal cellular proliferation | 0.0399 | Shh/Kdr/Foxp2/Wnt5a |
| tissue homeostasis | 0.0415 | Dram2/Mfsd2a/Cd38/Kdr/Pdk4/Muc2/Ccn2/Cep290 |
| neuron death | 0.0415 | Shh/Kdr/Hmox1/Mt1/Agt/Ccl3/Ngfr/Tfap2a/Wnt5a/Wnt1 |
| endothelial cell migration | 0.0465 | Shh/Kdr/Hmox1/Agt/Wnt5a/Stc1/Cyp1b1 |
| regulation of reactive oxygen species | 0.0465 | Agt/Ngfr/Tfap2a/Cdkn1a/Xdh/Cyp1b1 |
| metabolic process | | |

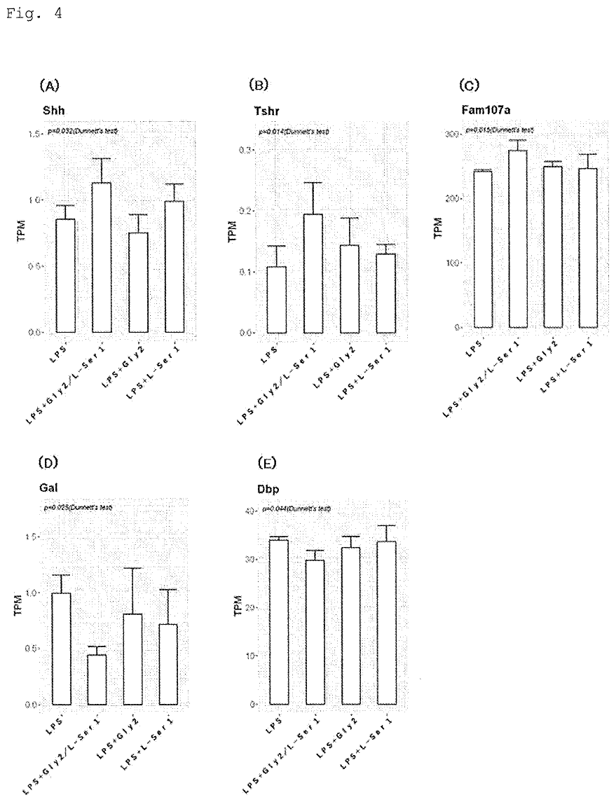

Among the genes whose expression levels were observed to vary in the hippocampus obtained from a mouse orally administered with the composition of Example 1, the expression levels of the Shh gene, Tshr gene, Gal gene, and Dbp gene, as well as the Fam107a gene involved in the memory function of the hippocampus, in the hippocampus obtained from each group are shown as mean+standard deviation in FIG. 4. Dunnett's test was performed between the respective groups of a group orally administered with the composition of Example 1 (LPS+Gly2/L-Ser1), a group orally administered with the agent of Comparative Example 4 (LPS+Gly2), and a group orally administered with the agent of Comparative Example 5 (LPS+L-Ser1), and a group orally administered with a solvent alone (LPS).

As shown in (A) and (B) of FIG. 4, among the up-regulated genes, the expression of the Shh gene, which is annotated for differentiation of dopaminergic neuron and regulation of nerve cell death, and the Tshr gene also annotated for differentiation of dopaminergic neuron, significantly increased (p<0.05) in the hippocampus obtained from the mouse of the group orally administered with the composition of Example 1 (LPS+Gly2/L-Ser1). As shown in FIG. 4(C), the expression of the Fam107a gene involved in the memory function of the hippocampus remarkably increased (significant at p<0.05) in the hippocampus obtained from the mouse of the group orally administered with the composition of Example 1 (LPS+Gly2/L-Ser1).

On the other hand, among the genes whose expression was down-regulated in the hippocampus obtained from the mouse of the group orally administered with the composition of Example 1 (LPS+Gly2/L-Ser1), as shown in FIGS. 4(D) and (E), the expression of the Gal gene encoding galanin, a type of neuropeptide, and the Dbp gene involved in synaptic plasticity in the hippocampus significantly decreased (p<0.05) in the hippocampus obtained from the mouse of the group orally administered with the composition of Example 1 (LPS+Gly2/L-Ser1).

As shown in FIGS. 4(A) to (E), such significant changes in the expression levels of genes were not observed in the hippocampus obtained from the mouse of the group orally administered with the agent of Comparative Example 4 (LPS+Gly2), and the hippocampus obtained from the mouse of the group orally administered with the agent of Comparative Example 5 (LPS+L-Ser1).

The above-mentioned results of Experimental Example 3 suggest that the composition of Example 1 had an effect of synergistically changing the expression of specific genes involved in the function of the hippocampus.

The gene groups whose expression increased in the hippocampus obtained from the mouse of the group orally administered with the composition of Example 1 (LPS+Gly2/L-Ser1) included multiple genes involved in memory function and hippocampal neuroprotection. Dopaminergic neurons, which were shown to be upregulated by GO analysis, are known to regulate a wide range of brain functions, including cognitive functions. The Wnt1 gene annotated in this GO term exhibits neuroprotective action by inhibiting the apoptotic pathway of neurons, and is attracting attention as an effective therapeutic target for Alzheimer's disease (L. Jia et al.; Mol. Brain 12, 104 (2019), which is incorporated herein by reference in its entirety). The Shh gene, whose expression level significantly increased in the hippocampus obtained from the mouse of the group orally administered with the composition of Example 1 (LPS+Gly2/L-Ser1) has been reported to afford neuroprotective action through multiple action mechanisms, including suppression of inflammation, promotion of angiogenesis and neurogenesis, and the like (L. Liu et al.; Neurochem. Res. 43 2199-2211 (2018), which is incorporated herein by reference in its entirety). Similarly, in mice in which the TShr gene, which is involved in the differentiation of dopaminergic neuron, was knocked out, spatial cognitive function and memory functions significantly decrease (S. Luan et al.; J. Endocrinol. 246 (1) 41-55 (2020), which is incorporated herein by reference in its entirety), and it has also been reported that increased expression of the Fam107a gene in the hippocampus improves cognitive function (M. V. Schmidt et al.; Proc. Natl. Acad. Sci. 108 (41) 17213-17218 (2011), which is incorporated herein by reference in its entirety).

On the other hand, it has been reported that the Gal gene, whose gene expression level was observed to be significantly decreased in the hippocampus obtained from the mouse of the group orally administered with the composition of Example 1 (LPS+Gly2/L-Ser1), inhibits cholinergic transmission in the hippocampus and decreases spatial memory function (S. E. Counts et al.; Cell Mol. Life Sci. 65 (12) 1842-1853 (2008), which is incorporated herein by reference in its entirety). Similarly, it has been clarified that the expression of the Dbp gene, whose gene expression level was observed to be decreased in the hippocampus obtained from the mouse of the group orally administered with the composition of Example 1 (LPS+Gly2/L-Ser1), is suppressed downstream of the neuroprotective action of the polypeptide GLP-1 produced in the intestine, and it has been reported that overexpression decreases spatial cognitive function (M. Klugmann et al.; Mol. Cell Neurosci. 31 (2):303-314 (2006), which is incorporated herein by reference in its entirety).

Therefore, the above-mentioned results in Experimental Example 3 suggest the possibility that oral administration of the composition of Example 1 significantly changed the expression of the gene group involved in memory function and neuroprotection and provided the memory function improving action suggested by the results in Experimental Example 1.

### INDUSTRIAL APPLICABILITY

As described in detail above, the present invention can provide a composition for improving cognitive function that can effectively prevent or improve a decline in cognitive function and cognitive dysfunction, and is highly safe and suitable for continuous ingestion or administration.

The composition for improving cognitive function of the present invention is particularly preferably used for the prevention or improvement of a decline in learning and/or memory functions and learning and/or memory disorders.

In addition, the composition for improving cognitive function of the present invention can increase the expression of multiple gene groups involved in neuroprotection of the hippocampus in addition to the memory function of the hippocampus, and thus can also act as a composition for improving or enhancing the neuroprotective function of the hippocampus.

Where a numerical limit or range is stated herein, the endpoints are included. Also, all values and subranges within a numerical limit or range are specifically included as if explicitly written out.

As used herein the words “a” and “an” and the like carry the meaning of “one or more.”

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A composition for improving cognitive function, comprising glycine or a salt thereof and serine or a salt thereof and having a content ratio of glycine to serine (glycine/serine) of not less than 2 in weight ratio.

2. The composition according to claim 1, further comprising one or more members selected from the group consisting of a phospholipid, glutamic acid or a salt thereof, cysteine or a salt thereof, and cystine or a salt thereof.

3. The composition according to claim 1, wherein the content ratio of glycine to serin (glycine/serine) is not more than 50 in weight ratio.

4. A cognitive function improving agent comprising a composition according to claim 1.

5. The agent according to claim 4, wherein the content ratio of glycine to serin (glycine/serine) is not more than 50 in weight ratio.

6. A food for improving cognitive function, comprising a composition according to claim 1.

7. The food according to claim 6, wherein the content ratio of glycine to serin (glycine/serine) is not more than 50 in weight ratio.

8. A composition for improving or enhancing neuroprotective function of the hippocampus, comprising glycine or a salt thereof and serine or a salt thereof and having a content ratio of glycine to serine (glycine/serine) of not less than 2 in weight ratio.

9. The composition according to claim 8, further comprising one or more members selected from the group consisting of a phospholipid, glutamic acid or a salt thereof, cysteine or a salt thereof, and cystine or a salt thereof.

10. A method for improving cognitive function, comprising administering to a subject in nee thereof an effective amount of a composition comprising glycine or a salt thereof and serine or a salt thereof and having a content ratio of glycine to serine (glycine/serine) of not less than 2 in weight ratio.

11. The method according to claim 10, wherein said composition further comprises one or more members selected from the group consisting of a phospholipid, glutamic acid or a salt thereof, cysteine or a salt thereof, and cystine or a salt thereof.

12. The method according to claim 10, wherein in said composition the content ratio of glycine to serin (glycine/serine) is not more than 50 in weight ratio.

13. The method according to claim 10, which is for improving a decline in learning and/or memory functions or learning and/or memory disorders.

14. The method according to claim 10, wherein said composition is in the form of a food.

15. The method according to claim 11, wherein said phospholipid is selected from the group consisting of phosphatidyl choline, phosphatidyl serine, phosphatidyl ethanolamine, phosphatidyl inositol, and phosphatidyl glycerol.

16. The method according to claim 11, wherein the total content of glycine and serine:total content of one or more members selected from the group consisting of a phospholipid, glutamic acid or a slat thereof, cysteine or a salt thereof, and cystine or a salt thereof is 10:1 to 1:10 in weight ratio.

17. The method according to claim 11, wherein the total content of glycine and serine:total content of one or more members selected from the group consisting of a phospholipid, glutamic acid or a slat thereof, cysteine or a salt thereof, and cystine or a salt thereof is 5:1 to 1:5 in weight ratio.

18. A method for improving or enhancing neuroprotective function of the hippocampus, comprising administering to a subject in need thereof an effective amount of a composition comprising glycine or a salt thereof and serine or a salt thereof and having a content ratio of glycine to serine (glycine/serine) of not less than 2 in weight ratio.

19. The method according to claim 18, wherein said composition further comprises one or more members selected from the group consisting of a phospholipid, glutamic acid or a salt thereof, cysteine or a salt thereof, and cystine or a salt thereof.
